(19) United States
(12) Patent Application Publication
(10) Pub. No.: US 2004/0208902 A1
(43) Pub. Date: Oct. 21, 2004

(54) Controlled-release nano-diffusion delivery systems for cosmetic and pharmaceutical compositions

(76) Inventor: Shyam K. Gupta, Scottsdale, AZ (US)

Correspondence Address: SHYAM K. GUPTA, BIODERM RESEARCH, 5221 E. WINDROSE DRIVE, SCOTTSDALE, AZ, 85254, US

(21) Appl. No.: 10/418,495

(22) Filed: Apr. 18, 2003

(51) Int. Cl. A61K 31/401; A61K 7/00

(52) U.S. Cl. 424/401; 514/423

(57) ABSTRACT

The present invention discloses the utilization of zeolites for controlled-release of cosmetic and pharmaceutical compositions by nano-diffusion technology. The treatment and protection of skin surface requires that certain compositions be delivered to the skin surface and allowed to remain on the skin surface for as long as possible before such ingredients are absorbed into deeper layers of skin and carried into the bloodstream. Zeolites do not absorb into the skin, which is useful for topical delivery of cosmetic and pharmaceutical compositions, for example antiaging, anti-wrinkle, antioxidants, skin whitening, acne treatment, rosacea treatment, sun screens, UV blocks, anesthetics, skin soothers, anti-irritants, anti-inflammatory agents, vitamins, hormones, and such that are electronically attached to the outer surfaces of such zeolites and are released to the outer surface of skin by a diffusion-controlled thermodynamic process.

### BACKGROUND OF INVENTION

A delivery system is a stratagem to enhance the efficacy or aesthetics of a cosmetic or pharmaceutical product. For example, the medicine part of a cough syrup is usually bitter in taste. The addition of sweeteners and flavor enhancers to such compositions makes them palatable. Sweetened and flavored cough syrups are thus delivery systems for the bitter medicine that is also included in such compositions.

For products desirable for topical delivery, there is usually a belief that the faster the absorption of such compositions into the skin the better they are. Although that may be applicable to certain compositions it is not universally desirable. For example, the protection of skin surface from harmful effects of UV and free-radicals requires that such protective compositions, such as sun screens and antioxidants, be delivered to the skin surface and allowed to remain on the skin surface for as long as possible before such ingredients are absorbed deeper into the skin and carried into the bloodstream. A number of other ingredients and compositions, such as topical anesthetics, skin whitening agents, topical antibacterials, anti-wrinkle agents, skin smoothing agents, hydroxy acids, and such can also benefit from such controlled topical delivery in elegant cosmetic compositions.

The prior art literature is abundant in the disclosures that improve, enhance, or accelerate the absorption of skin and body beneficial compositions into skin. There is a general lack of suitable compositions that can provide topical delivery of skin, body, and hair beneficial compositions which can remain on the skin surface for extended periods of time.

It is the purpose of the present invention to disclose a new technology for topical controlled-release of cosmetic and pharmaceutical compositions by nano-diffusion delivery systems based on zeolites.

Zeolites are a group of crystalline aluminosilicates that have a porous structure with a cavity. The preparation and properties of these zeolites are described in detail in U.S. Pat. No. 2,882,243, among other sources. Generally, the preparation involves combining aqueous solutions that are sources of silica, alumina and sodium to produce a gel which crystallizes upon hydrothermal treatment. Conventional washing and drying steps provide hydrated Zeolite Na. The hydrated Zeolite Na must be modified with the substitution of potassium for part of the sodium to form Zeolite K prior to activation. The potassium modification is carried out by ion exchange in aqueous solution using nearly any appropriate potassium salt such as potassium chloride, potassium nitrate, potassium sulfate, and the like. The exchange can be carried out in any convenient manner that allows control of the amount of potassium exchanged for sodium, or for sodium with other metals. Heating of the hydrated Zeolite K to a temperature above about 300° C. provides a zeolite that has a strong heat of hydration. Zeolites are alkaline materials, and in some personal care products such strong alkalinity is a disadvantage. In these products pH-adjusted zeolites with the proper potassium/sodium balance are useful. The pH adjustment is carried out after the potassium exchange. The exchanged zeolites are pH-adjusted by slurrying in water and adding acid slowly until the pH is between about 5.0 and 9.5. Mineral acids such as sulfuric acid and hydrochloric acid are usually used for this technique. The acidified slurry is aged for 30 to 90 minutes. Washing, filtering, drying and calcining complete the preparation. As can be noted, the preparation of such pH-adjusted zeolites is a cumbersome, multi-step process.

Zeolites have the following properties that can be highly useful for topical delivery of cosmetic and pharmaceutical compositions: (1) Zeolites have high adsorptive capacity for many organic compounds, and (2) Zeolites are available in certain pore sizes that can be used for self-warming or non-warming cosmetic and pharmaceutical compositions, and (3) Zeolites can be made anionic or cationic, which can be used for controlled-release of certain cosmetic and pharmaceutical ingredients via ion-pair mechanisms, and (4) Zeolites have a very large surface area that can nearly achieve a nano-particle distribution of organic molecules attached to its vast surface area, and (5) Zeolites can also be made in cations other than sodium or potassium, and (6) Zeolites do not absorb into the skin, which is useful for topical delivery of cosmetic and pharmaceutical compositions that are electronically attached to such zeolite surfaces.

However, many of the prior art applications of zeolites have centered upon their heat-releasing properties or trapping of ingredients inside their pore cavities. Zeolites also have outer surface area, in addition to such inner pore surface areas. The functional properties of zeolites utilizing such outer surface areas have not been utilized in the prior art, as shall become evident further.

Zeolites can be made with both specific pore structures and bound cations that have found applications in various self-warming cosmetic compositions. U.S. Pat. No. 3,250,680 (Menkart et al.) discloses applications of Zeolites for the preparation of self-heating toothpaste and other such compositions. This utilizes only the heat releasing property of zeolites.

U.S. Pat. No. 4,626,550 (Hertzenberg) discloses certain personal care products such as lotions and creams that are prepared using Zeolite A that contains sodium and potassium. Further, pH-adjusted zeolite A to provide a less basic material for these personal care products was prepared by heating after the Zeolite A has had its sodium-potassium ratio adjusted. It is not convenient to prepare such less alkaline zeolites due to high temperature required for calcination of such pH-adjusted zeolites.

U.S. Pat. No. 4,379,143 (Sherry et al.) discloses activated or partially activated zeolites that can be included in analgesic balms or ointments as improved replacements for rubefacients. Upon hydration, the zeolite becomes warm, thereby helping to relieve pains associated with various musculoskeletal problems. Sherry et al. thus utilize only the heat-releasing property of zeolites and do not disclose any controlled-release applications.

U.S. Pat. No. 6,274,128 (Bergman et al.) discloses an essentially anhydrous hair conditioning composition comprising zeolites of specific pore size larger than the critical diameter of a water molecule and both the carrier molecules and the hair conditioner molecules that have molecular diameters larger than the largest average pore size of the micro porous materials. As is clearly evident, such constraints are not convenient or commercially achievable at a reasonable cost.

U.S. Pat. No. 6,309,655 (Minnix) discloses a cosmetic composition comprising a self-heating component, self-indicating disintegrating granules comprised of water-insoluble polymer and a colorant, which gives users indications of the length of time the composition has been applied and the degree of mixing when in use. This application is thus aimed at self-heating properties of zeolites, and their length of heating effect.

U.S. Application 20010016201 (Janchitraponvej) discloses a yet another self-heating application of an anhydrous rinse-out hair care composition utilizing zeolites.

Self-warming compositions have also been made with various anhydrous alkali metal salts (Giani et al., U.S. Pat. No. 5,747,004). Such compositions do not provide as much heat as the zeolite-based compositions, and they are also highly alkaline.

Another approach to such self-warming systems is the use of a redox-system, based on iron powder and carbon (Gott et al., U.S. Pat. No. 6,287,580). Such compositions are not suitable for topical cosmetic applications, as they will discolor skin surface.

In self-warming formulations based on Zeolites, the pore size specification is typically very small, from 3 to 10 angstroms in diameter, as is the ratio between sodium and potassium cations bound to silicate anions of such zeolites. These formulations release heat upon contact with water. Water penetrates the pores of such Zeolites and hydrates the interior silicate atoms of Zeolite agglomerates. Such interaction of zeolite with water releases the heat of hydration. Most cosmetic lotion, cream, shampoo, and conditioner products also contain hydrophilic and lipophilic ingredients for skin and hair care benefits. Some of such ingredients tend to clog the pores of Zeolites, causing a reduction in the heat-release properties of such formulations. The examples of such fatty materials that can inhibit the heat release properties of zeolites include most surfactants used in shampoo and body wash applications; quaternary ammonium compounds used for hair conditioning applications; fatty esters used as emollients in skin lotion and cream applications, and other similar examples. In prior art, these problems have been solved by the use of small pore size zeolites that permit the entrance of water molecules inside their cavity but not other larger size molecules, for example U.S. Pat. No. 6,274,128. This limits the applicability of zeolites of larger pore size in self-heating cosmetic preparations. Additionally, the Zeolites, in contact with water, typically show an alkaline pH. For example, zeolite “Siliporite” (from Atofina Corporation) gave the following pH in deionized water suspension: 1% w/w Siliporite pH 10.8; 5% w/w Siliporite pH 11.3; and 25% Siliporite pH 12.1. Such high pH is not compatible with healthy skin's normal pH range, which is slightly acidic (pH 5.2 to 6.5). If such high pH products are applied to sensitive skin areas, such as face or scalp, then skin irritation or dry skin conditions can develop. It would be advantageous to formulate such self-warming compositions in the non-alkaline, skin compatible pH zone. However, as noted earlier, the preparation of pH-balanced zeolites requires several steps and is not convenient.

Zeolites can also be made in cations other than sodium or potassium. U.S. Pat. No. 6,084,142 (Yao et al.) discloses the preparation of a zinc zeolite, and its application in petroleum cracking process. U.S. Pat. No. 6,177,374 (Pradhan et al.) discloses the preparation of silicon, zinc and aluminum zeolites, and their application in petroleum cracking process. Yao and Pradhan do not disclose any cosmetic applications of such zeolite derivatives. U.S. Pat. No. 6,479,427 (Anthony at al.) and U.S. Pat. No. 5,502,240 (Pugach) disclose titanium zeolites and their application in petroleum cracking process. U.S. Pat. No. 5,772,917 (Kynast et al.) discloses a cesium zeolite that is luminescent. U.S. Pat. No. 6,106,797 (Muller et al.) discloses titanium or vanadium zeolites useful for accelerating oxidation reactions. U.S. Pat. No. 6,008,389 (Grosch et al.) discloses titanium and vanadium zeolites useful as catalysts for the preparation of epoxides, in particular propylene oxide, from olefins, hydrogen and oxygen. U.S. Patent Application 20030035763 (Vergani et al.) discloses the use of iron and manganese zeolites in the purification of organometallic compounds utilizing such zeolite's adsorptive properties. U.S. Patent Application 20030024856 (Surana et al.) discloses a yet another application of zeolite's adsorptive properties in removing odors. U.S. Patent Application 20020127402 (Green et al.) discloses the antimicrobial applications of silver ions attached to zeolites by ion-exchange methods. The attachment of any organic molecules to zeolites by ion-exchange method has not been disclosed by Green et al.

It is worthy of note that although zeolites with many different cations, such as titanium, zinc, manganese, iron, and copper have been disclosed, any applications of such metal zeolites in pharmaceutical applications have not been disclosed. It is further worthy of note that both titanium and zinc are well known in their oxide state as sun block agents that have been used in sunscreen compositions now for several years. It would thus be of much potential commercial and consumer interest to develop applications of titanium and zinc zeolites in cosmetic or pharmaceutical compositions. It is yet another point worthy of note that zeolites with their sodium or potassium ions exchanged with organic anions and cations have not been disclosed so far, since such organic molecules bound by ion-exchange process with zeolite's silicate backbone could provide controlled-release properties for their extended efficacy, lowered skin irritation, lowered toxicity, and more uniform topical bioavailability, among other such cosmetic and pharmaceutical benefits.

This lack of knowledge is of special importance, since zeolites with enhanced ion-exchange capacity are well known (U.S. Patent Application 20010053741, Mikko et al.; U.S. Pat. No. 5,935,891; Prior). U.S. Pat. No. 6,503,740 (Alther et al.) discloses zeolites treated with an organic modification compound such as quaternary amines, pyridinium compounds, and phosphonium amines that are useful for water treatment applications. U.S. Pat. No. 6,365,130 (Barry et al.) discloses zeolites exchanged with antimicrobial metals for a chewing gum application, or a laundry application (U.S. Pat. No. 6,454,813; Chan). Modified zeolites have been used for topical cancer therapy (U.S. Pat. No. 6,288,045; Kaufman).

### SUMMARY OF INVENTION

The present invention discloses the utilization of zeolites for controlled-release of cosmetic and pharmaceutical compositions by nano-diffusion technology. The treatment and protection of skin surface requires that certain compositions be delivered to the skin surface and allowed to remain on the skin surface for as long as possible before such ingredients are absorbed into deeper layers of skin and carried away into the bloodstream. Zeolites do not absorb into the skin, which is useful for topical delivery of cosmetic and pharmaceutical compositions, for example antiaging, anti-wrinkle, antioxidants, skin whitening, acne treatment, rosacea treatment, sun screens, UV blocks, anesthetics, skin soothers, anti-irritants, anti-inflammatory agents, vitamins, hormones, and such that are electronically attached to the outer surfaces of such zeolites and are released to the outer surface of skin by a diffusion-controlled thermodynamic process.

The present invention centers also on the utilization of the following properties of various Zeolites for the topical delivery of cosmetic and pharmaceutical compositions:

(1) Zeolites have high adsorptive capacity for many organic compounds, both inside and outside of their pore cavity surface areas, and (2) Zeolites are available in certain pore sizes that can be used for self-warming or non-warming cosmetic and pharmaceutical compositions, and (3) Zeolites can be made anionic or cationic, which can be used for controlled-release of certain cosmetic and pharmaceutical ingredients via ion-pair mechanisms, and (4) Zeolites have a very large surface area that can potentially achieve a nano-particle distribution of organic molecules attached to its vast surface area, and (5) Zeolites can also be made in cations other than sodium or potassium, and (6) Zeolites do not absorb into the skin, which is useful for topical delivery of cosmetic and pharmaceutical compositions that are electronically attached to outer surface of such zeolites.

Based on the above properties of Zeolites, the following applications of various Zeolites in cosmetic and pharmaceutical compositions are still unknown: (1) The delivery of anionic, cationic, and amphoteric cosmetic and pharmaceutical compositions in controlled-release topical applications via ion-pair mechanisms, and (2) The delivery of non-ionic cosmetic and pharmaceutical compositions in controlled-release topical applications via adsorption or pore-trapping mechanisms, and (3) The delivery of other cations, such as titanium and zinc, on the skin surface for protection from UVA and UVB, and (4) The controlled-release of heat from self-warming Zeolite compositions that do not require specific sodium to potassium cation ratio, or certain specific pore size specifications of such Zeolites, and (5) The adjustment of the pH of alkaline or acidic Zeolites to match skin's own pH for topical applications of such Zeolites that does not require a high temperature calcination process.

It is the intention of the present invention to disclose the novel applications that utilize Zeolite's above mentioned attributes.

The present invention discloses certain self-heating and non-heating body, skin, and hair care cosmetic compositions that, in contrast to the prior art examples mentioned herein, do not require the following: (1) Zeolites of any specific pore size, and (2) A specific balance of sodium and potassium cations, and (3) Limitations on the use of ingredients and compositions that may clog the pore of Zeolites and reduce its heat releasing power, and (4) pH balancing of alkaline Zeolites that does not require high temperature calcinations process.

### DETAILED DESCRIPTION

Zeolites have a very large surface area that is ionic in its nature. This surface area covers both the outside of zeolite and the inside zeolite's porous cavity. The size of the pores of this cavity determines the size of any molecules that can enter zeolite's internal cavity. Almost all prior art disclosures have focussed on the cavity of zeolite. Since molecules larger in size than zeolite's cavity can not enter zeolite's internal surface area, the delivery of such molecules from zeolite has not been disclosed in the prior art.

I have now found that zeolite's outer surface area can be used for the controlled delivery of molecules that are too large to fit inside zeolite's cavity. Moreover, the delivery of such molecules can be controlled by a diffusion-controlled delivery process to provide a sustained-release of such molecules for long term benefits. It is theorized at this point that the ionic nature of zeolite's outer surface binds with many molecules in various modes such as ionic bond, ion-pair bonding, electrostatic attraction, Van der Waal's attraction forces, or Hydrogen bonding. Upon contact with the outer layers of skin such molecules diffuse from zeolite surface to skin surface. This is because skin's outer surface has both positively charged and negatively charged centers (from basic and acidic amino acids that are present in skin's protein structure) which have a stronger affinity for such molecules. This results in an overall controlled-release delivery of such molecules from zeolite surface. Although the controlled-release delivery of skin and body beneficial ingredients and compositions has been practiced for a long time by using solid, the surface area of such controlled-release materials, for example starch and talc, is very limited and not as large as that of zeolites. In practical terms, zeolites provide surface area approaching that of nano-particles.

The present invention thus also provides the first application of zeolites as nano-delivery system particles in which the delivery of cosmetic and pharmaceutical ingredients is performed by nano-diffusion (i.e. the ingredients and compositions are delivered to skin surface by diffusion as single atoms or molecules from the large surface area of zeolites to which such ingredients and compositions are attached by various binding mechanisms mentioned above).

The binding of skin beneficial molecules or compositions to zeolite surface is best performed in the presence of a solubilizer composition. The solubilizer composition has the capacity to solubilize skin beneficial molecule or composition. However, it is not always necessary to solubilize such compositions first. The ingredients that are deposited on zeolite surface as particles or micro-droplets can also be delivered in the controlled-release manner.

A great variety of skin beneficial ingredients and compositions can be deposited and bound to the outer surface of zeolites. The examples include antiaging, anti-wrinkle, antioxidants, trace metal peptides, trace metal nucleotides, trace metal glycosides, body slimming, fat reduction agents, skin whitening, acne treatment, rosacea treatment, sun screens, UV blocks, anesthetics, skin soothers, anti-irritants, anti-inflammatory agents, vitamins, hormones, alkaloids, and such.

Even the ingredients that are difficult to formulate in cosmetic compositions, or are unstable, can be formulated in the compositions of the present invention. The examples include, but not limited to, Retinol, Retinoic acid, Retene A, Phytantriol, Panthenol, Thiamine, Riboflavin, Niacin, Niacinamide, Vitamin C, Vitamin C derivatives, Pyridoxine, Biotin, Folic Acid, Coenzyme Q10, Lipoic Acid, and Hydroquinone.

The preparation of cosmetic and pharmaceutical compositions with controlled-release delivery mode can be achieved by several methods, all of which are simple to operate with commonly available manufacturing equipment. The ingredients of various compositions are simply mixed together. In some cases, some heating is required. This is necessary only in order to melt down the solid components of such compositions for better mixing. The skin and body beneficial ingredients and compositions are attracted to zeolite surface and are held by various mode of attachment, such as ionic charges, ion-pair, Van der Waal's forces, Hydrogen bonding, and such. It is not necessary that such ingredients and compositions penetrate and enter zeolite cavity. In fact, such inner cavity entrapments can actually retard the efficacy of certain compositions due to their inability to exit such cavity once they are trapped inside. This is due to very strong ionic interactions (it is much like an insect trapped inside a spider's web). In this regard, the present invention depends largely on the attachment of ingredients and compositions on the vast surface of zeolite, which is quite unlike prior art disclosures that depend on the entrapment of ingredients and compositions inside the porous cavity of zeolite.

Relative to various ingredients and compositions that can be attached by various mechanisms to zeolite surface; there is virtually no limit to such materials. The zeolite is also not limited to any specific pore size, molecular dimension, cation ratios, or particle size. The finer the particle size of zeolite the better the nano-diffusion. This is because finer the zeolite greater its active surface area that can bind with skin beneficial ingredients and compositions.

### EXAMPLES

The following examples are presented to illustrate presently preferred practice thereof. As illustrations they are not intended to limit the scope of the invention. All quantities are in weight %.

### Example 1

### An Anhydrous Face Mask Controlled-Release Antiaging Composition with Heat-releasing Effect.

(1) Magnesium Sulfate (Anhydrous) 30.0

(2) Glycerin 49.0

(3) Sodium Potassium Aluminosilicate (Zeolite A3) 20.0

(4) Antiaging Composition 1.0 (The antiaging composition is an equal weight mixture of Tetrahydrocurcumin, Niacinamide Lactate, Copper ATP complex, Glutathione, and Carnosine).

Procedure: All ingredients are mixed in a dry atmosphere. A white paste is obtained. The face is rinsed with water first, then the mask composition is applied as a film. The heat is felt immediately.

### Example 2

### This Example Shows the Formulation of a Self-Warming Scrubbing Facial Mask Product that Contains a Large Number of Hydrophilic and Lipophilic Ingredients for Controlled-Release Skin Benefits

(1) Glycerine 44.7

(2) Dimethicone 2.0

(3) Vitamin A Palmitate 0.1

(4) Vitamin E Acetate 0.1

(5) Phenoxyethanol 0.5

(6) Antileukine 0.1

(7) Phycocorail 0.1

(8) Codiavelane 0.1

(9) Aosaine 0.1

(10) Bio Structure 0.1

(11) Bio Extender 0.1

(12) Liquapar 0.2

(13) Fragrance 0.2

(14) Zeolite (Atofina Nk30np) 30.0

(15) Huber 90 White Clay 14.0

(16) Ascorbic Acid 0.5

(17) Aleuritic Acid 0.5

(18) Mandelic Acid 0.5

(19) Veegum 2.0

(20) Fine Grain Salt 4.0

(21) Dead Sea Salt 0.1

Procedure. All ingredients are mixed in a dry atmosphere. A white paste is obtained.

### Example 3

### This example shows the formulation of a self-heating body, hair and facial wash product with controlled-release antiaging ingredients.

(1) PEG-6 33.5

(2) Vitamin A Palmitate 0.1

(3) Vitamin E Acetate 0.1

(4) Actiplex Botanicals 0.1

(5) Phenoxyethanol 0.5

(6) Liquapar 0.2

(7) Niacinamide 0.5

(8) Zeolite (Atofina Nk30np) 37.0

(9) Sodium Lauryl Sulfoacetate 8.5

(10) Sodium Cocoyl Isethionate 14.0

(11) Citric Acid 4.0

(12) Fragrance 0.5

Procedure: Mix all ingredients in a homogenizer mill. A paste is obtained.

### Example 4

### This Example Shows the Formulation of a Self-Warming Hair Conditioner Cosmetic Product With Hair Strengthening Ingredients

(1) Incroquat Behenyl TMS 4.5

(2) Zeolite (Atofina Nk30np) 25.5

(3) PEG-6 67.5

(4) Lactic Acid 0.5

(5) Citric Acid 2.0

Procedure: Mix all ingredients and heat at 60 to 70C. Cool with mixing, making sure moisture is kept out. A pearly white paste is formed.

### Example 5

### Self-Heating Body Butter with Skin Whitening Ingredients

(1) Castor Oil 20.8

(2) Mango Butter 2.0

(3) Cocoa Butter 4.0

(4) Beeswax 3.5

(5) Stimu-Tex 0.2

(6) Avocado Butter 1.0

(7) Shea Butter 4.0

(8) Sweet Almond Oil 2.0

(9) Grapeseed Oil 2.0

(10) Dimethicone 5.0

(11) Hydrogenated Soybean Oil 6.0

(12) Sesame Oil 0.9

(13) Tinoguard TT 0.2

(14) Phenoxyethanol 0.5

(15) Propyl Paraben 0.2

(16) Aloe Vera (In Oil) 4.0

(17) Vitamin E Acetate 0.1

(18) Vitamin A Palmitate 0.1

(19) Zeolite (Atofina Nk30np) 35.0

(20) Lactic Acid 5.0

(21) Fragrance 3.5

Procedure: Mix all ingredients and heat at 60 to 70C. Cool to room temperature. A butter-like material is obtained.

### Example 6

### This Example Shows the Formulation of a Self-Warming Anti-Acne Facial Wash

(1) Glycerine 22.288

(2) Methylpropanepdiol 15.0

(3) Vitamin A Palmitate 0.001

(4) Vitamin E Acetate 0.001

(5) Actiplex 0.01

(6) Phenoxyethanol 0.5

(7) Liquapar 0.2

(8) Niacinamide Lipoate 0.5

(9) Zeolite Atofina Nk30np) 37.0

(10) Citric Acid 3.0

(11) Sodium Lauryl Sulfoacetate 7.0

(12) Veegum 1.0

(13) Sodium Cocoyl Isethionate 12.0

(14) Fragrance 1.5

Procedure: Mix all ingredients and heat at 50 to 60C. Cool to room temperature. A paste-like product is obtained.

### Example 7

### This Example Shows the Formulation of a Self-Warming Facial Anhydrous Mud Mask Product with Anti-Wrinkle Ingredients

(1) PEG-6 45.0

(2) Dimethicone 2.0

(3) Vitamin A Palmitate 0.001

(4) Vitamin E Acetate 0.001

(5) Actiplex 0.01

(6) Phenoxyethanol 0.5

(7) Liquapar 0.2

(8) Zeolite (Atofina Nk30np) 36.0

(9) Huber 90 White Clay 14.0

(10) Veegum 2.0

(11) Copper ATP 0.1

(12) Glutathione 0.1

(13) Licorice Root Extract 0.5%

Procedure: Mix all ingredients at 50 to 60C. Cool to room temperature. A paste4ike material is obtained.

### Example 8

### Self-Heating Facial Clay Composition with Anti-wrinkle and Anti-Oxidant Ingredients

(1) Glycerin 18.748

(2) Methylpropanediol 22.0

(3) Dimethicone 2.0

(4) Vitamin A Palmitate 0.001

(5) Vitamin E Acetate 0.001

(6) Dehydrated Aloe 0.01

(7) Cactus Extract 0.01

(8) Orange Extract 0.01

(9) Yucca Extract 0.01

(10) Prickly Pear Fruit Extract 0.01

(11) Fragrance 0.5

(12) Phenoxyethanol 0.5

(13) Liquapar 0.2

(14) Zeolite (Thermilux) 36.0

(15) Lactic Acid 0.5

(16) Citric Acid 2.5

(17) Glycolic Acid 0.5

(18) Malic Acid 0.5

(19) Huber 90 White Clay 14.0

(20) Veegum 2.0

Procedure: Mix all ingredients at 40 to 45C. Cool to room temperature. A paste-like composition is obtained.

### Example 9

### The Product Obtained in example 7 (1 Gram) was Mixed with Deionized Water (99 Grams), and the pH of the Mixture was Found to be 6.5

### Example 10

### Self-Warming Shampoo. This Example Shows the Use of Niacin (Nicotinic Acid) as the Acid Activator Ingredient

(1) Glycerine 48.0

(2) Vitamin A Palmitate 0.1

(3) Vitamin E Acetate 0.1

(4) Actiplex 2794 (Plant Extracts) 0.1

(5) Phenoxyethanol 0.5

(6) Liquapar 0.2

(7) Niacin 0.5

(8) Zeolite (WR Grace Silosiv) 30.0

(9) Sodium Lauryl Sulfoacetate 10.0

(10) Sodium Cocoyl Isethionate 10.0

(11) Fragrance 0.5

Procedure: Mix all ingredients in a homogenizer. A paste-like product is obtained.

### Example 11

### Non-Heat Releasing Facial Mask with 10% AHA and 0.4% BHA

(1) Deionized water 48.29

(2) Dimethicone 4.0

(3) Vitamin A Palmitate 0.01

(4) Vitamin E Acetate 0.5

(5) Phenoxyethanol 0.5

(6) Liquapar 0.2

(7) Zeolite (Atofina Nk30np) 36.0

(8) Glycolic Acid (AHA) 5.0

(9) Lactic Acid (AHA) 2.5

(10) Mandelic Acid (AHA) 2.5

(11) Salicylic Acid (BHA) 0.5

Procedure: Mix 1 To 6. Add 8, 9, 10 and 11 to it and mix. Add 7 to it and mix. A paste-like product is obtained.

### Example 12

### How to Determine Amount of Organic Acid Required in a Formulation to Neutralize the Alkalinity of Zeolites for a Skin Compatible pH Range

Suspend 1 gram of warming component in 99 grams of deionized water.

Mix for 15 minutes. Determine its pH with a pH meter. Add a water-soluble organic acid gradually to it till pH 5.5 to 6.5 is reached. The amount of organic acid needed to neutralize warming component from the weight of organic acid required for this neutralization is calculated according to Equation 1.

Weight acid required for warming component=Weight of warming component X Weight of organic acid needed for above pH adjustment (Equation 1)

For example, in one experiment, 0.1 grams of citric acid was required for 1.0 grams of zeolite in 99 grams of deionized water. For 50 grams of zeolite clay in a formulation,

Weight citric acid required for 50 grams of warming clay=50×0.1=5.0 grams

This is an approximate calculation, as other components in the formulation can affect this stoichiometry.

I claim:

1. A cosmetic or pharmaceutical composition comprising:
(i) at least one zeolite composition, and
(ii) at least one skin beneficial composition requiring a controlled-release topical delivery, and
(iii) a cosmetically or pharmaceutically acceptable delivery system, or a carrier base composition.

2. A cosmetic or pharmaceutical composition comprising:
(i) From about 1% to about 70% of at least one zeolite composition, and
(ii) From about 0.0001% to about 50% of at least one skin beneficial composition, and
(i) From about 20% to about 99% of a cosmetically or pharmaceutically acceptable delivery system, or a carrier base composition.

3. A composition according to claim 1, wherein zeolite is selected from a group of aluminosilicates that can be either in hydrated or anhydrous forms.

4. A composition according to claim 1, wherein skin beneficial cosmetic or drug composition can be selected to provide treatment of skin aging, skin wrinkles reduction, skin exfoliating, antioxidants, collagen and elastin synthesis boosters, various hydroxy acids (alpha hydroxy acids, beta hydroxy acids, and polyhydroxy acids), vitamins, hormones, skin whitening agents, UVA/UVB sunscreens, antimicrobial agents, antifungal agents, treatment of acne, treatment of rosacea, age-spots reduction, skin surface brightening, striae distensae (stretch marks) reduction, treatment of pimples, treatment of skin infections and lesions, varicose and spider veins reduction, blood microcirculation improvement, UVA/UVB protection of skin, skin redness reduction benefits, excess body fat reduction, cellulite control, body slimming, body toning, or combinations thereof.

5. A composition according to claim 1, wherein a cosmetically acceptable delivery system or a carrier base can be selected in the form of a lotion, cream, gel, spray, thin liquid, body splash, mask, serum, solid cosmetic stick, lip balm, shampoo, liquid soap, bar soap, bath oil, cologne, hair conditioner, salve, collodion, impregnated patch, impregnated strip, skin surface implant, and any other such cosmetically or pharmaceutically acceptable topical delivery forms.

6. The compositions according to claim 1, wherein the cosmetically or pharmaceutically acceptable delivery system can be traditional water and oil emulsions, suspensions, colloids, microemulsions, clear solutions, suspensions of nanoparticles, emulsions of nanoparticles, or anhydrous compositions.

7. A composition according to claim 1, wherein cosmetically or pharmaceutically acceptable delivery system or carrier base can optionally include additional skin beneficial ingredients selected from skin cleansers, surfactants (cationic, anionic, non-ionic, amphoteric, and zwitterionic), skin and hair conditioning agents, vitamins, hormones, minerals, plant extracts, anti-inflammatory agents, concentrates of plant extracts, emollients, moisturizers, skin protectants, humectants, silicones, skin soothing ingredients, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof. The quantities of such ingredients can be safe and effective amounts as needed, and not limited to any specific limits.

8. A composition according to claim 4, wherein the composition to promote excess fat reduction, cellulite control, or toning benefits can be selected from the group consisting of Forskohlin extract (from Coleus forskohlii plant), Hydroxycitric acid, (from Garcinia cambogia, and plants of Garcinia family), L-Carnitine, Creatine, Human growth hormone (HGH), Chromium picolinate, Kola seed extract, Caffeine, Niacinamide, Psyllium husk, Chitosan, Lipoprotein complexes, Polyphenols, Gymnemic acid, Pyruvic acid and Pyruvate salts, salts of Hydroxycitric acid, Phaseolamin (from Phaseolus vulgaris extract), DHEA, Chitosan, Theophylline, Theobromine (or salts thereof such as Aminophylline), Roselle tea extract, Arabinose, Inosine, Adenosine, Fructose-1,6-diphosphate, Adenosine triphosphate (ATP), Adenosine diphosphate (ADP), Indomethacin, Baicalein, Extract of the plant of genus Tephrosia, Natriuretic peptide, Laminaria extract, Extract from berries of Panax genus plant, Gymnema sylvestre extract, 9-cis, 1-trans Conjugated linoleic acid and 10-trans, 12-cis conjugated linoleic acid isomers (conjugated linoleic acid, CLA), Synephrine, Hordenine, Octopamine, Tyramine, N-Methyltyramine, Azaftig, Extract of Climbing ivy (Hedera helix), Extract of Arnica (Arnica montana), Extract of Rosemary (Rosmarinus officinalis), Extract of Marigold (Calendula officinalis), Extract of Sage (Salvia officinalis), Extract of Ginseng (Panax ginseng), Extract of St. Johns-wart (Hypericum perforatum), Extract of Ruscus (Ruscus aculeatus), Extract of meadowsweet (Filipendula ulmaria), Extract of Orthosiphon (Ortosifon stamincus), and combinations thereof.

9. A composition according to claim 4, wherein the composition to promote collagen and elastin in the skin can be selected from Ascorbic acid, Ascorbic acid derivatives, Glucosamine ascorbate, Arginine ascorbate, Lysine ascorbate, Glutathione ascorbate, Nicotinamide ascorbate, Niacin ascorbate, Allantoin ascorbate, Creatine ascorbate, Creatinine ascorbate, Chondroitin ascorbate, Chitosan ascorbate, DNA Ascorbate, Carnosine ascorbate, Vitamin E, various Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), α-Lipoic acid, Niacinamide lipoate, Glutathione, Andrographolide (Andrographis paniculata), Carnosine, Niacinamide, Potentilla erecta extract, Polyphenols, Grapeseed extract, Pycnogenol (Pine Bark extract), and combinations thereof. The quantities of such compositions can be safe and effective amounts as needed, and not limited to any specific limits.

10. A composition according to claim 4, wherein the skin beneficial hydroxy acid can be selected from the group consisting of salicylic acid, lactic acid, glycolic acid, malic acid, mandelic acid, ascorbic acid, ascorbyl phosphoric acid, hydroxycitric acid, hydroxytetronic acid, citric acid, aleuritic acid, ellagic acid, rosmarinic acid, chlorogenic acid, polysulfonic acid, and hyaluronic acid (HYA). The quantities of such compositions can be safe and effective amounts as needed, and not limited to any specific limits.

11. A composition according to claim 4, wherein the skin beneficial skin whitening agent can be selected from hydroquinone, arbutin, hydroquinone derivatives, Paper Mulberry extract (Broussonetia kazinoke), Mitracarpe extract (Mitracarpus scaber), Bearberry extract (Arctostaphylos uva ursi), Yellow Dock extract (Rumex crispus and Rumex occidentalis), Glutathione, Leucocyte extract, Aspergillus orizae extract (Aspergillus orizae), Licorice Root extract (Glycyrrhiza glabra), Rosmarinic acid (Rosmarinus officinalis), Tetrahydrocurcumin, Green Tea extract (Camellia sinensis), Yohimbe extract (Pausinystalia yohimbe), Ecklonia cava extract, niacinamide, Hydroxytetronic acid, Spondias mombin extract, Maprounea guianensis extract, Walteria indica extract, Gouania blanchetiana extract, Cordia schomburgkii extract, Randia armata extract, Hibiscus furcellatus extract, and combinations thereof. The quantities of such compositions can be safe and effective amounts as needed, and not limited to any specific limits.

12. A composition according to claim 4, wherein the skin beneficial antioxidant composition can be selected from Ascorbic acid, Ascorbic acid derivatives, Vitamin E, Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), alpha-Lipoic acid, Glutathione, Andrographolide, Grapeseed extract, Green Tea Extract, Polyphenols, Pycnogenol (Pine Bark extract), White Tea extract, Black Tea extract, (Andrographis paniculata), Carnosine, Niacinamide, Emblica extract, and combinations thereof. The quantities of such compositions can be safe and effective amounts as needed, and not limited to any specific limits.

13. A composition according to claim 4, wherein the UVA/UVB sunscreen composition can be selected from Titanium dioxide, Zinc oxide, Galanga extract (Kaempferia galanga), Benzophenone-3, Benzophenone-4, Ethylhexyl Methoxycinnamate, Homosalate, Ethylhexyl salicylate, Octocrylene, Menthyl anthranilate, Avobenzone, Lawsone, Sulisobenzone, Trolamine salicylate, Lawsone, Glyceryl aminobenzoate, Cinoxate, and PABA. and combinations thereof. The quantities of such compositions can be safe and effective amounts as needed, and not limited to any specific limits.

14. A Composition According to claim 4, wherein the blood microcirculation improvement composition is selected from Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Sericoside, Visnadine, Thiocolchicoside, Grapeseed Extract, Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis-Vinifera) leaves, apigenin, phytosan, luteolin, and combinations thereof. The quantities of such compositions can be safe and effective amounts as needed, and not limited to any specific limits.

15. A composition according to claim 4, wherein the antimicrobial composition can be selected from Berberine, Triclosan, Triclocarban, various Tritons (quaternary ammonium compounds), Benzyl Alcohol, Dehydroacetic Acid, Phenoxyethanol, and combinations thereof. The quantities of such compositions can be safe and effective amounts as needed, and not limited to any specific limits.

16. A composition according to claim 4, wherein the vitamin composition can be selected from Vitamin A, Retinol, Retinoic acid, Tretinoin, members of Vitamins B group, Vitamin C, Vitamin D, Vitamin E, Vitamin K, Carotenes, Biotin, Folic Acid, and their derivatives, and combinations thereof. The quantities of such ingredients can be safe and effective amounts as needed, and not limited to any specific limits.

17. A composition according to claim 4, wherein the hormone composition can be selected from progesterone, androsterone, dehydroepiandrosterone (DHEA), Pregnenolone, androstenedione, melatonin, testosterone, and combinations thereof. The quantities of such compositions can be safe and effective amounts as needed, and not limited to any specific limits.

18. A composition according to claim 4, wherein the skin protectant drug composition can be selected from Allantoin, petrolatum, glycerin, dimethicone, urea, calamine, cocoa butter, kaolin, zinc acetate, zinc carbonate, and combinations thereof. The quantities of such compositions can be safe and effective amounts as needed, and not limited to any specific limits.

19. A composition according to claim 4, wherein skin beneficial ingredient can be selected from various trace metal delivery systems, which includes copper, zinc, and manganese in their both free and chelated forms.
